(19) United States
(12) Patent Application Publication
(10) Pub. No.: US 2013/0018288 A1
(43) Pub. Date: Jan. 17, 2013

(54) SYSTEM FOR MONITORING ONGOING CARDIOPULMONARY RESUSCITATION

(71) Applicant: Michael Brian Jaffe, Cheshire, CT (US)

(72) Inventor: Michael Brian Jaffe, Cheshire, CT (US)

(73) Assignee: KONINKLIJKE PHILIPS ELECTRONICS N.V., Eindhoven (NL)

(21) Appl. No.: 13/635,919

(22) PCT Filed: Mar. 17, 2011
(86) PCT No.: PCT/IB2011/051121

§ 371 Date: Sep. 19, 2012

Related U.S. Application Data
(60) Provisional application No. 61/317,998, filed on Mar. 26, 2010.

(51) Int. Cl. A61H 31/00; A61M 16/00

(52) U.S. Cl. 601/41

(57) ABSTRACT

Cardiopulmonary resuscitation being provided to a subject is monitored. An enhanced measurement of the effectiveness of the cardiopulmonary resuscitation received by the subject is determined by correlating chest compressions with changes in movement and/or composition of gas at or near the airway of the subject. For example, one or more therapy parameters may be measured with an enhanced accuracy and/or precision, and/or one or more therapy parameters not monitored in conventional cardiopulmonary resuscitation monitoring systems may be measured.

### BACKGROUND OF THE INVENTION

1. Field of the Invention

The invention relates to monitoring the effectiveness of cardiopulmonary resuscitation in real time (or near real time) to provide caregivers with feedback related to the effectiveness of the cardiopulmonary resuscitation.

2. Description of the Related Art

Systems configured to monitor the effectiveness of cardiopulmonary resuscitation are known. Typically, these systems monitor the chest of a subject receiving cardiopulmonary resuscitation to determine the timing of chest compressions and/or the displacement to the chest during chest compressions. Conventional systems may implement information (e.g., impedance information) detected at the chest of the subject to determine information about gas in the lungs of the patient.

### SUMMARY OF THE INVENTION

One aspect of the invention relates to a system configured to monitor cardiopulmonary resuscitation. In one embodiment, the system comprises a gas sensor, a motion sensor, and a processor. The gas sensor is configured for placement in fluid communication with an airway of a subject. The gas sensor is further configured generate an output signal conveying information related to one or more gas parameters of gas at or near the airway of the subject. The motion sensor is configured to generate an output signal conveying information related to motion of the chest of the subject. The processor is configured to determine a therapy parameter related to the effectiveness of cardiopulmonary resuscitation received by the subject based on the output signal of the gas sensor and the output signal of the motion sensor.

Another aspect of the invention relates to a method of monitoring cardiopulmonary resuscitation. In one embodiment, the method comprises generating a first output signal conveying information related to one or more gas parameters of gas at or near the airway of the subject; generating a second output signal conveying information related to motion of the chest of the subject; and determining a therapy parameter related to the effectiveness of cardiopulmonary resuscitation received by the subject based on the first output signal and the second output signal.

Yet another aspect of the invention relates to a system configured to monitor cardiopulmonary resuscitation. In one embodiment, the system comprises means for generating a first output signal conveying information related to one or more gas parameters of gas at or near the airway of the subject; means for generating a second output signal conveying information related to motion of the chest of the subject; and means for determining a therapy parameter related to the effectiveness of cardiopulmonary resuscitation received by the subject based on the first output signal and the second output signal.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. In one embodiment of the invention, the structural components illustrated herein are drawn to scale. It is to be expressly understood, however, that the drawings are for the purpose of illustration and description only and are not a limitation of the invention. In addition, it should be appreciated that structural features shown or described in any one embodiment herein can be used in other embodiments as well. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 illustrates a system configured to monitor cardiopulmonary resuscitation, in accordance with one or more embodiments of the invention.

FIG. 2 illustrates a method of monitoring cardiopulmonary resuscitation, according to one or more embodiments of the invention.

### DETAILED DESCRIPTION OF THE EXEMPLARY EMBODIMENTS

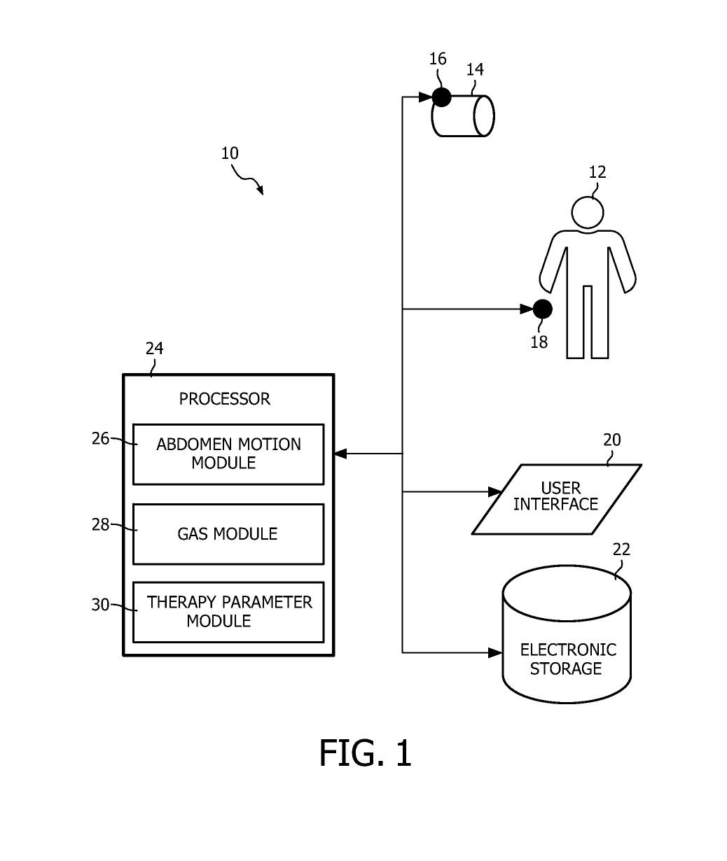

FIG. 1 illustrates a system 10 configured to monitor cardiopulmonary resuscitation being provided to a subject 12. The system 10 is configured to provide an enhanced measurement of the effectiveness of the cardiopulmonary resuscitation received by subject 12. For example, system 10 may measure one or more therapy parameters with an enhanced accuracy and/or precision, and/or may measure one or more therapy parameters not monitored in conventional cardiopulmonary resuscitation monitoring systems, and/or may provide other enhancements. In one embodiment, system 10 includes one or more of a subject interface appliance 14, one or more gas sensors 16, one or more chest motion sensors 18, a user interface 20, electronic storage 22, one or more processors 24, and/or other components.

The subject interface appliance 14 is configured to provide gas to and/or receive gas from one or more external orifices of the airway of subject 12. As such, subject interface appliance 14 forms the conduit by which gas is forced into the airway (and into the lungs) of subject 12 during cardiopulmonary resuscitation. In one embodiment, subject interface appliance 14 is coupled to a resilient “bag” that is squeezed by a caregiver to force gas within the bag through subject interface appliance 14 and into the airway of subject 12. In one embodiment, subject interface appliance 14 is coupled to a mouthpiece through which a caregiver blows gas through subject interface appliance 14 and into the airway of subject 12. The subject interface appliance 14 may include one or more of an endotracheal tube, a nasal cannula, a tracheotomy tube, a nasal mask, a nasal/oral mask, a full face mask, a total face mask, a partial rebreathing mask, or other interface appliances that communicate a gas with an airway of a subject.

The gas sensor 16 is configured for placement in fluid communication with the airway of subject 12. The gas sensor 16 is placed in fluid communication with the airway of subject 12 via subject interface appliance 14. As such, gas coming out of the airway of subject 12 (e.g., being exhaled by subject 12) is received by subject interface appliance 14, and at least a portion of this gas is provided to gas sensor 16. In one embodiment, gas sensor 16 is disposed within a sidestream sampling system that receives gas from subject interface appliance 14. In one embodiment, gas sensor 16 is disposed within a mainstream sampling system that receives gas from subject interface appliance 14.

The gas sensor 16 is configured to generate one or more output signals convey information related to one or more gas parameters of gas at or near the airway of subject 12. By way of non-limiting example, the one or more gas parameters may include one or more of flow, pressure, composition (e.g., partial pressure, concentration, etc.) of one or more molecular species of respiratory gases (e.g., carbon dioxide, oxygen, nitrogen, etc.), temperature, humidity, trace gas measurements, therapeutic gas measurements, and/or other gas parameters.

Although gas sensor 16 is shown in FIG. 1 as being a single body or device, this is for illustrative purposes only, and is not intended to be limiting. The gas sensor 16 may include a plurality of sensors that generate output signals conveying information related to gas parameters. In one non-limiting embodiment, gas sensor 16 includes a sensor that generates an output signal conveying information related to gaseous composition, and a sensor that generates an output signal conveying information related to flow. Where gas sensor 16 includes a plurality of sensors, the sensors may be substantially co-located (as is shown in FIG. 1), or may be disposed at relatively disparate physical locations to receive gas from the airway of subject 12 via subject interface appliance 14.

The chest motion sensor 18 is configured to generate one or more output signals conveying information related to motion of the upper chest of subject 12 while receiving cardiopulmonary resuscitation. Such information may include the position of one or more surfaces of the chest, the velocity of one or more surfaces of the chest, the acceleration of one or more surfaces of the chest, circumference around the chest, and/or other information related to the motion of the chest during cardiopulmonary resuscitation.

In one embodiment chest motion sensor 18 is to be physically carried on or by the chest of subject 12 during cardiopulmonary resuscitation, and to generate the one or more output signals based on its own position (and/or changes to its position). For example, chest motion sensor 18 may include an accelerometer carried within a device configured to be placed on the chest of subject 12 during subject 12. In one embodiment, chest motion sensor 18 is configured to detect motion of the upper chest of subject 12 in a non-contact manner. For example, chest motion sensor 18 may include an imaging sensor configured to capture an image of (or including) the chest of subject 12, an optical sensor, an ultrasonic sensor, and/or other non-contact position/motion sensors.

The user interface 20 is configured to provide an interface between system 10 and a caregiver (e.g., the individual(s) administering cardiopulmonary resuscitation through which the caregiver may provide information to and/or receive information from system 10. This enables data, results, and/or instructions and any other communicable items, collectively referred to as “information,” to be communicated between the caregiver and system 10. Examples of interface devices suitable for inclusion in user interface 20 include a keypad, buttons, switches, a keyboard, knobs, levers, a display screen, a touch screen, speakers, a microphone, an indicator light, an audible alarm, and a printer. In one embodiment, the functionality of which is discussed further below, user interface 20 actually includes a plurality of separate interfaces.

It is to be understood that other communication techniques, either hard-wired or wireless, are also contemplated by the present invention as user interface 20. For example, the present invention contemplates that user interface 20 may be integrated with a removable storage interface provided by electronic storage 22. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 20 include, but are not limited to, an RS-232 port, RF link, an IR link, modem (telephone, cable or other). In short, any technique for communicating information with system 10 is contemplated by the present invention as user interface 20.

In one embodiment, electronic storage 22 comprises electronic storage media that electronically stores information. The electronic storage media of electronic storage 22 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 22 may store software algorithms, information determined by processor 24, information received via user interface 20, and/or other information that enables system 10 to function properly. Electronic storage 22 may be a separate component within system 10, or electronic storage 22 may be provided integrally with one or more other components of system 10 (e.g., processor 24).

The processor 24 is configured to provide information processing capabilities in system 10. As such, processor 24 may include one or more of a digital processor, an analog processor, a digital circuit designed to process information, an analog circuit designed to process information, a state machine, and/or other mechanisms for electronically processing information. Although processor 24 is shown in FIG. 1 as a single entity, this is for illustrative purposes only. In some implementations, processor 24 may include a plurality of processing units. These processing units may be physically located within the same device, or processor 24 may represent processing functionality of a plurality of devices operating in coordination. For example, in one embodiment, the functionality attributed below to processor 24 is divided between one or more first processors that are carried by subject interface appliance 14 and/or a unit that also carries gas sensor 16 (e.g., a device placed on the chest of subject 12 during cardiopulmonary resuscitation), and a second processor that is carried by a separate device (e.g., a base unit).

As is shown in FIG. 1, processor 24 may be configured to execute one or more computer program modules. The one or more computer program modules may include one or more of a motion module 26, gas module 28, therapy module 30, and/or other modules. Processor 24 may be configured to execute modules 26, 28, and/or 30 by software; hardware; firmware; some combination of software, hardware, and/or firmware; and/or other mechanisms for configuring processing capabilities on processor 38.

It should be appreciated that although modules 26, 28, and 30 are illustrated in FIG.

1 as being co-located within a single processing unit, in implementations in which processor 24 includes multiple processing units, one or more of modules 26, 28, and/or 30 may be located remotely from the other modules. The description of the functionality provided by the different modules 26, 28, and/or 30 described below is for illustrative purposes, and is not intended to be limiting, as any of modules 26, 28, and/or 30 may provide more or less functionality than is described. For example, one or more of modules 26, 28, and/or 30 may be eliminated, and some or all of its functionality may be provided by other ones of modules 26, 28, and/or 30. As another example, processor 24 may be configured to execute one or more additional modules that may perform some or all of the functionality attributed below to one of modules 26, 28, and/or 30.

The motion module 26 is configured to determine information related to motion of the chest of subject 12 during cardiopulmonary resuscitation. Such information is determined based on the output signal(s) generated by chest motion sensor 18. The information determined by motion module 26 may include, for example, one or more of acceleration of a surface of the chest of subject 12 (e.g., the chest of subject 12), a velocity of surface of the chest of subject 12, a position of the chest of subject 12, a circumference of the chest of subject 12, and/or other information related to the motion of the chest of subject 12 as the chest of subject 12 is compressed during cardiopulmonary resuscitation. The motion module 26 is configured to determine such information in an ongoing manner (e.g., at a predetermined sampling rate) so that the information may determined by motion module 26, or considered in subsequent processing, as a function of time.

The gas module 28 is configured to determine one or more gas parameters of gas at or near the airway of subject 12. The one or more gas parameters are determined based on the output signal generated by gas sensor 16. By way of non-limiting example, the one or more gas parameters may include one or more of concentration of a molecular species of respiratory gases (e.g., carbon dioxide, oxygen, nitrogen, etc.), partial pressure of a molecular species of respiratory gases, pressure, flow rate, temperature, humidity, trace gas measurements, therapeutic gas measurements, and/or other gas parameters. The gas module 28 is configured to determine the one or more parameters in an ongoing manner (e.g., at a predetermined sampling rate) so that the one or more parameters are provided by processor 24, or considered in subsequent processing, as a function of time.

The therapy module 30 is configured to determine one or more therapy parameters related to the effectiveness of cardiopulmonary resuscitation received by the subject. The determination of the therapy parameter(s) by therapy module 30 is based on both the output signal of the gas sensor 16 and the output signal of the chest motion sensor 18. In one embodiment, therapy module 30 determines a therapy parameter from information determined by motion module 26 and/or one or more gas parameters determined by gas module 28. In one embodiment, therapy module 30 determines a therapy parameter directly from the output signal generated by gas sensor 16 and/or from the output signal generated by chest motion sensor 18. The one or more therapy parameters may include one or more of respiratory rate, end-tidal carbon dioxide partial pressure, a volumetric capnograpy measurement, a therapy parameter that quantifies the effectiveness of cardiopulmonary resuscitation by the depth of changes to the end-tidal carbon dioxide partial pressure waveform corresponding to chest compressions, a tidal volume, volumetric oxygraphy measurement, cardiac output, surrogates and/or derivations based on these parameters, volumetric deadspace, and/or other parameters.

The determination of the one or more therapy parameters based on both of the output signal generated by gas sensor 16 and the output signal generated by chest motion sensor 18 enhances the accuracy and/or precision of the therapy parameter(s), facilitates the determination of one or more therapy parameters that may not otherwise be readily determined automatically, and/or may provide other enhancements over determinations of parameters related to cardiopulmonary resuscitation using only one or the other of the output signals generated by gas sensor 16 and chest motion sensor 18. These enhancements are a result, at least in part, of the impact of chest compressions during cardiopulmonary resuscitation on the flow of gas within the respiratory system of subject 12. Assuming the airway of subject 12 is unblocked, chest compressions result in the expulsion of gas from the lungs of subject 12. Such gas includes alveolar gas that has a higher carbon dioxide content than ambient atmosphere. At least some of the alveolar gas passes through the airway of subject 12, and is exhaled from subject 12. As a chest compression ends, the lungs of subject 12 expand, resulting are from ambient atmosphere being drawn into the airway (and lungs) of subject 12.

The output signal generated by gas sensor 16 reflects the presence of alveolar gas being exhaled from the lungs during chest compression in the form of elevated levels of carbon dioxide. By correlating momentary rises in carbon dioxide (and/or flow) with chest compressions and/or the corresponding breaths caused by chest compressions, therapy module 30 enhances the determination of the one or more therapy parameters.

In one embodiment, therapy module 30 is further configured to provide feedback (other than the therapy parameter(s)) to a caregiver about the cardiopulmonary resuscitation being provided to subject 12. This feedback is based on analysis of the one or more therapy parameters that are determined.

For example, in one embodiment, the therapy module 30 is further configured to compare the volume of gas inhaled and/or exhaled by subject 12 during respiration with the volumetric deadspace (the airway of subject 12 between the alveoli and ambient atmosphere). Based on this comparison, therapy module 30 determines whether additional respiration support is needed (e.g., via forcing air into the airway of subject 12 by way of subject interface appliance 14), or if the inhalation and exhalation caused by chest compressions are sufficient. The therapy module 30 may determine additional information about the additional respiratory support needed by subject 12 based on the comparison of the volume of gas inhaled and/or exhaled with the volumetric deadspace. These determinations are then provided to the caregiver (e.g., via user interface 20).

The determination of whether additional respiration is needed, the timing at which the additional respiration is required, and/or other aspects of the feedback provided to the caregiver may be determined by comparing the volume of gas in excess of the volumetric deadspace that is being inhaled and/or exhaled by subject 12. For example, above a first threshold, therapy module 30 may determine that no additional respiratory support is required. Between the first threshold and a second threshold, therapy module 30 may determine that air should be forced into the lungs of subject 12 at a first rate (e.g., 1 breath for 15 compressions). Between the second threshold and a third threshold, therapy module 30 may determine that air should be forced into the lungs of subject 12 at a second rate (e.g., 2 breaths for 15 compressions). This example is not intended to be limiting. For example, therapy module 30 may implement more of less thresholds in this manner.

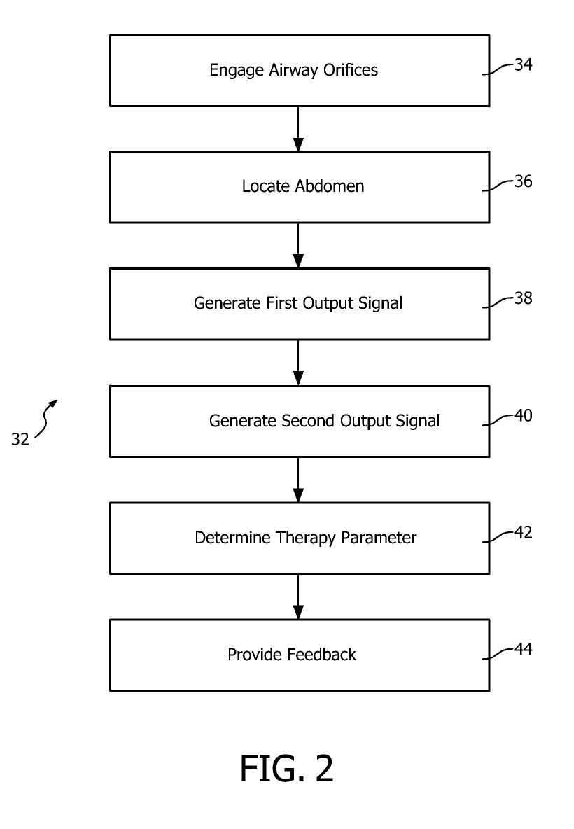

FIG. 2 illustrates a method 32 of monitoring cardiopulmonary resuscitation. The operations of method 32 presented below are intended to be illustrative. In some embodiments, method 32 may be accomplished with one or more additional operations not described, and/or without one or more of the operations discussed. Additionally, the order in which the operations of method 32 are illustrated in FIG. 2 and described below is not intended to be limiting.

In some embodiments, method 32 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 32 in response to instructions stored electronically on an electronic storage medium. The one or more processing devices may include one or more devices configured through hardware, firmware, and/or software to be specifically designed for execution of one or more of the operations of method 32.

At an operation 34, one or more external orifices of an airway (e.g., nostrils and/or mouth) of a subject are engaged to provide gas to, and receive gas from, the airway of the subject. In one embodiment, operation 34 is performed by a subject interface appliance similar to or the same as subject interface appliance 14 (shown in FIG. 1 and described above).

At an operation 36, one or more surfaces of the chest of the subject are located for monitoring motion of the chest of the subject. This may include physically engaging a surface of the chest of the subject (e.g., the chest). In one embodiment operation 36 is performed by an chest motion sensor similar to or the same as chest motion sensor 18 (shown in FIG. 1 and described above), or a device carrying the same.

At an operation 38, a first output signal is generated conveying information related to one or more gas parameters of gas at or near the airway of the subject. The first output signal is generated based on gas received from the one or more external orifices of the airway engaged at operation 34. The one or more gas parameters may include relative concentration of a molecular species, a partial pressure of a molecular species, pressure, flow rate, and/or other parameters. In one embodiment, operation 38 is performed by a gas sensor similar to or the same as gas sensor 16 (shown in FIG. 1 and described above).

At an operation 40, a second output signal is generated conveying information related to motion of the chest of the subject. The second output signal is generated based on information related to the one or more surfaces of the chest that are acquired at operation 36. The information related to motion of the chest of the subject may include, for example, the position, velocity, and/or acceleration of one or more surfaces of the chest of the subject, the circumference of the chest of the subject, and/or other information. In one embodiment, operation 40 is performed by an chest motion sensor similar to or the same as chest motion sensor 18 (shown in FIG. 1 and described above).

At an operation 42, a therapy parameter is determined. The therapy parameter is related to the effectiveness of cardiopulmonary resuscitation received by the subject. The therapy parameter is determined at operation 42 based on the first output signal and the second output signal. In one embodiment, operation 42 is performed by a therapy module similar to or the same as therapy module 30 (shown in FIG. 1 and described above).

At an operation 44, feedback about the ongoing cardiopulmonary resuscitation is provided to one or more caregivers. The feedback may include the therapy parameter determined at operation 42, and/or may be based on the therapy parameter. The feedback may be determined by a therapy module similar to or the same as therapy module 30 (shown in FIG. 1 and described above). The feedback may be provided to the one or more caregivers by a user interface similar to or the same as user interface 20 (shown in FIG. 1 and described above).

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system configured to monitor cardiopulmonary resuscitation, the system comprising:
a gas sensor configured for placement in fluid communication with an airway of a subject, the gas sensor being further configured to generate an output signal conveying information related to one or more gas parameters of gas at or near the airway of the subject;
a motion sensor configured to generate an output signal conveying information related to motion of a chest of the subject; and
a processor configured (a) to determine a therapy parameter related to an effectiveness of cardiopulmonary resuscitation received by the subject based on both (a)(i) the output signal of the gas sensor and (a)(ii) the output signal of the motion sensor and (b) to provide feedback, via a user interface, about the cardiopulmonary resuscitation being provided to the subject, wherein the feedback is based on an analysis of one or more therapy parameters that are determined.

2. The system of claim 1, wherein the gas sensor is configured such that the output signal generated by the gas sensor conveys information related to composition of gas at or near the airway of the subject.

3. The system of claim 1, wherein the therapy parameter comprises one or more of respiratory rate, volumetric deadspace, and/or end tidal carbon dioxide.

4. The system of claim 1, further comprising a subject interface appliance configured to provide gas to, and receive gas from, one or more external orifices of the airway of the subject, and wherein the sensor is configured to communicate with the airway of the subject via the subject interface appliance.

5. The system of claim 1, wherein the motion sensor comprises an accelerometer.

6. A method of monitoring cardiopulmonary resuscitation, the method comprising:
generating, via a gas sensor configured for placement in fluid communication with an airway of a subject, a first output signal conveying information related to one or more gas parameters of gas at or near the airway of the subject;
generating, via a motion sensor, a second output signal conveying information related to motion of a chest of the subject; and
determining, via a processor, (a) a therapy parameter related to an effectiveness of cardiopulmonary resuscitation received by the subject based on both (a)(i) the first output signal and (a)(ii) the second output signal and (b) providing feedback, via a user interface, about the cardiopulmonary resuscitaton being provided to the subject, wherein the feedback is based on an analysis of one or more therapy parameters that are determined.

7. The method of claim 6, wherein the first output signal conveys information related to composition of gas at or near the airway of the subject.

8. The method of claim 6, wherein the therapy parameter comprises one or more of respiratory rate, volumetric deadspace, and/or end tidal carbon dioxide.

9. The method of claim 6, further comprising engaging one or more external orifices of the airway of the subject to provide gas to, and receive gas from, the engaged one or more external orifices of the airway of the subject, and wherein the first output signal is generated based on measurements of gas provided to and/or received from the engaged one or more external orifices of the airway of the subject.

10. The method of claim 6, wherein the second output signal conveys information related to acceleration of the chest of the subject.

11. A system configured to monitor cardiopulmonary resuscitation, the system comprising:
means for generating a first output signal conveying information related to one or more gas parameters of gas at or near the airway of a subject;
means for generating a second output signal conveying information related to motion of a chest of the subject; and
means for determining a therapy parameter related to an effectiveness of cardiopulmonary resuscitation received by the subject based on both (a)(i) the first output signal and (a)(ii) the second output signal and (b) providing feedback, via a user interface, about the cardiopulmonary resuscitation being provided to the subject, wherein the feedback is based on an analysis of one or more therapy parameters that are determined.

12. The system of claim 11, wherein the first output signal conveys information related to composition of gas at or near the airway of the subject.

13. The system of claim 11, wherein the therapy parameter comprises one or more of respiratory rate, volumetric deadspace and/or end tidal carbon dioxide.

14. The system of claim 11, further comprising means for engaging one or more external orifices of the airway of the subject to provide gas to, and receive gas from, the engaged one or more external orifices of the airway of the subject, and wherein the means for generating the first output signal communicates with the airway of the subject via the means for engaging.

15. The system of claim 11, wherein the second output signal conveys information related to acceleration of the chest of the subject.
